(19) United States
(12) Patent Application Publication
(10) Pub. No.: US 2009/0192577 A1
(43) Pub. Date: Jul. 30, 2009

(54) MEDICAL ELECTRICAL LEAD WITH COATED CONDUCTOR

(76) Inventor: Shrojalkumar Desai, Little Canada, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/250,865

(22) Filed: Oct. 14, 2008

Related U.S. Application Data
(60) Provisional application No. 61/024,079, filed on Jan. 28, 2008.

(51) Int. Cl. A61N 1/05

(52) U.S. Cl. 607/116

(57) ABSTRACT

A medical electrical lead includes a conductor having at least one layer of a parylene coating formed from a polyxylylene based polymer. The conductor can be a cable or a coiled conductor including one or more individual conductive filaments. The parylene coating may be provided over the individual conductive filaments or provided over an outer periphery of the conductor formed from the individual conductive filaments. Additionally, the parylene coating can be provided in more than one location. Lead bodies having reduced outer diameters without compromising the desired physical properties of a medical electrical lead may be constructed.

### TECHNICAL FIELD

The present invention relates to medical electrical leads. More particularly, the present invention relates to medical electrical lead having coated conductors.

### BACKGROUND

Implantable medical devices for treating a variety of medical conditions with electrical stimuli are well known. Implantable medical devices generally include a medical electrical lead for delivering an electrical stimulus to a targeted site within a patient's body such as for example, a patient's heart or nervous system. Such leads generally have an elongated, flexible insulating body, one or more inner conductors extending through lumens formed in the body and one or more exposed electrodes connected to the distal ends of the conductors.

Leads may be introduced into the patient's vasculature at a venous access site and travel through veins to the sites where the leads' electrodes will be implanted or otherwise contact tissue at the targeted therapy site. A power source attached to the proximal ends of the conductors delivers an electrical stimulus therapy to the targeted site via the one or more conductors.

Important characteristics of the medical electrical leads include biocompatibility, durability and reduced size. However, it has been an ongoing challenge to manufacture medical electrical leads possessing all of these characteristics. One particular challenge has been the prevention of lead degradation caused by metal ion migration and/or oxidation originating from the lead conductor(s).

### SUMMARY

According to one embodiment a medical electrical lead includes: a lead body including a proximal end configured to be connected to a pulse generator and a distal end; at least one conductor extending from the proximal end to the distal end of the lead body, and at least one electrode operatively connected to the at least one conductor. The conductor may be, for example, a coiled conductor or a cable conductor. In some embodiments, the lead may include multiple conductors of different types. According to various embodiments, the conductor is coated with parylene or a derivative thereof. The parylene coating is non-porous and substantially pin-hole free, and capable of conforming to the surface features of the surface onto which it is disposed.

According to one embodiment, the parylene coating is disposed over individual conductive wires or filaments used in constructing the conductor such that the individual wires or filaments are separately coated. According to another embodiment, the parylene coating is disposed over an outer surface of at least a portion of the conductor. According to other embodiments, the parylene coating is disposed of the entire outer surface of the conductor extending from substantially the proximal end to the distal end of the lead body. According to yet another embodiment, the parylene coating is disposed between an inner coiled conductor and an outer coiled conductor in a coiled conductor having a co-axial configuration.

According to other embodiments, the parylene coating is disposed over the individual components of an extendable/retractable fixation mechanism. According to yet other embodiments, the parylene coating coats the inner circumference of the lumen from which an extendable/retractable fixation mechanism extends and retracts.

According to another embodiment of the present invention, a medical electrical lead includes: a lead body including a proximal end configured to be connected to a pulse generator and a distal and having an outer diameter ranging from about 2 to about 15 French; at least one coiled conductor operatively coupled to a first electrode; and at least one cable conductor operatively coupled to a second electrode.

According to one embodiment, at least one coiled conductor includes: one or more individual conductive filaments; a first layer of a coating comprising parylene or a parylene derivative provided over the individual conductive filaments such that the individual conductive filaments are separately insulated from one another, the first layer of parylene coating being non-porous and substantially pin-hole free and having a thickness ranging from about 0.1 μm to about 100 μm. According to further embodiments, the coiled conductor includes a second layer of a parylene coating comprising parylene or a derivative thereof provided over an outer surface of the at least one coiled conductor, the second layer of parylene coating being non-porous and substantially pin-hole free and having a thickness ranging from about 0.1 μm to about 100 μm.

According to another embodiment, at least one cable conductor includes a first layer of a parylene coating comprising parylene or a derivative thereof provided over the individual conductive filaments such that the individual conductive filaments are separately insulated from one another, the first layer of parylene coating being non-porous and substantially pin-hole free and having a thickness ranging from about 0.1 μm to about 100 μm. According to further embodiments, the at least one cable conductor includes a second layer of a parylene coating comprising parylene or a derivative thereof provided over an outer surface of the at least one coiled conductor, the second layer of parylene coating being non-porous and substantially pin-hole free and having a thickness ranging from about 0.1 μm to about 100 μm.

According to various further embodiments, the parylene coatings are capable of conforming to the surface features of the surface of the substrate on which the coating is disposed.

According to a yet further embodiments of the present invention, the parylene coating includes Parylene N or derivatives thereof.

According to other further embodiments of the present invention, the parylene coating includes can be Parylene C, D, or HT or derivatives thereof.

According to yet further embodiments of the present invention, the parylene coating includes an FDA approved parylene or parylene derivative.

According to yet another further embodiment, the at least one coiled conductor has a co-radial configuration.

According to yet another further embodiment, the at least one coiled conductor has a co-axial configuration including an inner coiled conductor disposed within an outer second coiled conductor and a third layer of parylene coating comprising a poly-p-xylylene polymer provided over the outer surface of inner coiled conductor such that it insulates and separates the inner coiled conductor from the outer coiled conductor.

According to some embodiments of the present invention, the poly-p-xylylene polymer has a dielectric strength of greater than about 5,000 (Volts/mil). According to other embodiments of the present invention, the poly-p-xylylene polymer has a moisture vapor transmission rate of equal to or less than about 1.75 g-mil/100 in².

### BRIEF DESCRIPTION OF THE DRAWINGS

FIG. 1 is a schematic view of a medical electrical lead according to an embodiment of the present invention.

FIG. 2A is side schematic view of a portion of a lead body including a coiled conductor having at least one conductive filament according to an embodiment of the present invention.

FIG. 2B is a end, cross-sectional view of the conductive filament shown in FIG. 2A according to an embodiment of the present invention.

FIG. 3 is an end, cross-sectional view of a lead body according to an embodiment of the present invention.

FIG. 4 is an end, cross-sectional view of a lead body according to another embodiment of the present invention.

FIG. 5 is a side schematic view of a portion of a conductor according to an embodiment of the present invention.

FIG. 6 is a side schematic view of a portion of a conductor according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In the following detailed description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

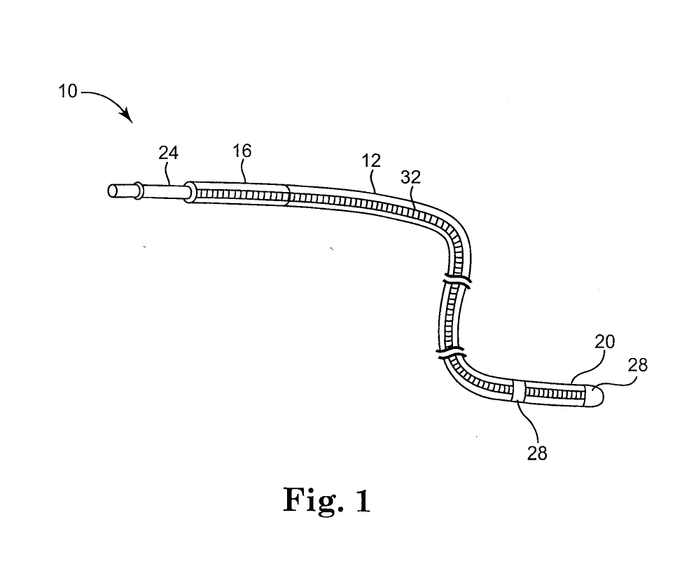

FIG. 1 is a partial cross-sectional view of a medical electrical lead 10, according to an embodiment of the present invention. Medical electrical lead 10 includes an elongated flexible lead body 12 extending from a proximal end 16 to a distal end 20. The proximal end 16 is configured to be operatively connected to a pulse generator via a connector 24. At least one conductor 32 extends from the connector 24 at the proximal end 16 of the lead 10 to one or more electrodes 28 at the distal end 20 of the lead 10. In further embodiments, the lead body 12 includes a lumen adapted to receive a guiding element such as a guidewire or a stylet for delivery of the lead 10 to a target location within a patient's heart.

The lead body 12 is flexible, but substantially non-compressible along its length, and has a circular cross-section. According to one embodiment of the present invention, an outer diameter of the lead body 12 ranges from about 2 to about 15 French. According to other embodiments of the present invention, an outer diameter of the lead body is less than about 10 French. The medical electrical lead 10 may be unipolar, bipolar, or multi-polar depending upon the type of therapy to be delivered. In embodiments of the present invention employing multiple electrodes 28 and multiple conductors 32, each conductor 32 is adapted to be connected to an individual electrode 28 in a one-to-one manner allowing each electrode 28 to be individually addressable.

The electrodes 28 can have any electrode configuration as is known in the art. According to one embodiment of the present invention, at least one electrode can be a ring or partial ring electrode. According to another embodiment, at least one electrode 28 is a shocking coil. According to yet another embodiment of the present invention, at least one electrode 28 includes an exposed electrode portion and an insulated electrode portion. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

According to various embodiments, the lead body 12 can include one or more fixation members for securing and stabilizing the lead body 12 including the one or more electrodes 28 at a target site within a patient's body. The fixation member(s) can be active or passive. In some embodiments, the fixation member can be a screw-in fixation member. In other embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,463,334 which is herein incorporated by reference.

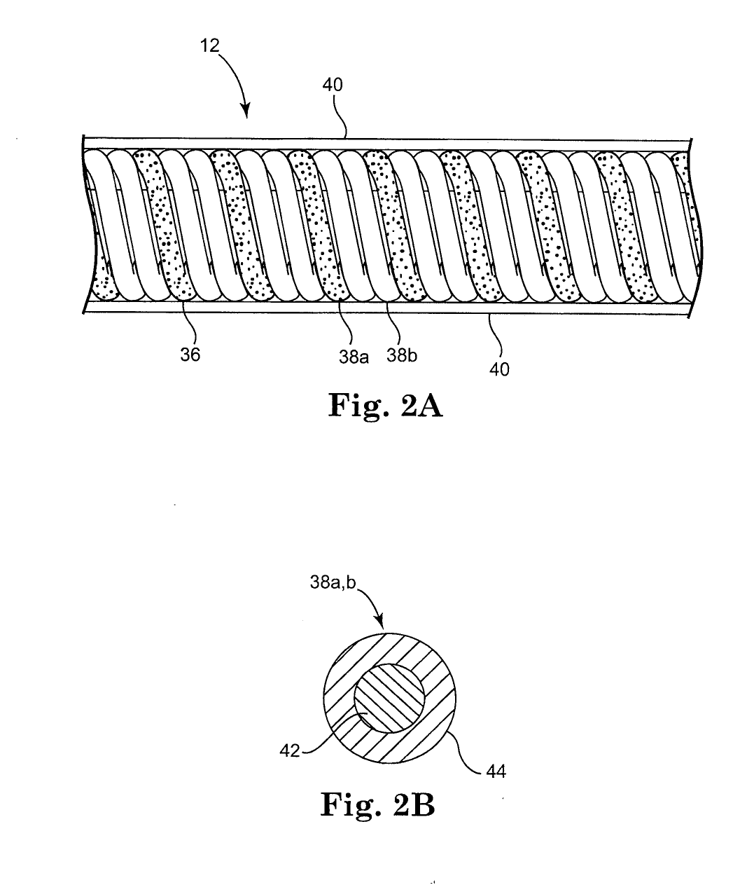

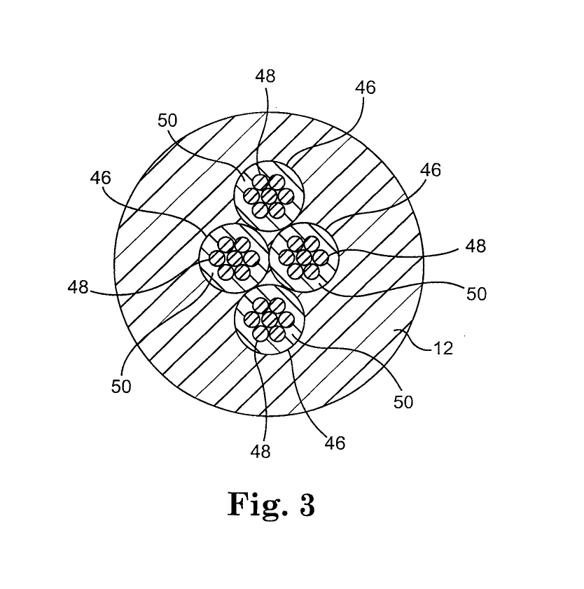

FIGS. 2A-6 show the conductor 32 according to various embodiments of the present invention. The conductor 32 may be a coiled conductor, as a shown in FIG. 2A, or a cable conductor, as shown in FIG. 3. A coiled conductor is generally helical in configuration and includes one or more conductive wires or filaments. A cable conductor has a substantially linear configuration and can also include a plurality of conductive wires or filaments.

In each embodiment illustrated in FIGS. 2-6, a coating formed from parylene or a derivative thereof is disposed over one or more conductors 32 or conductive filaments. The parylene coating is a conformal coating in that it substantially conforms to the surface features of the surface on which it is disposed. Additionally, the parylene coating is non-porous and free from pin holes.

“Parylene” is a generic name for a known group of poly-p-xylylene polymers. Poly-p-xylylene polymers and derivatives thereof typically have a repeating structure of

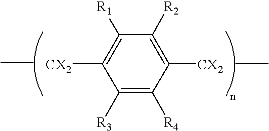

wherein X is a halogen or a hydrogen, and R₁, R₂, R₃, R₄ are each independently a hydrogen, a halogen, an alkyl, an alkyl halide, amino, nitro, alkylamine, alkyl hydroxy, or an alkyl carboxy group, and n is at least 2.

Two commercially available forms of parylene include Parylene N and Parylene C. Parylene N is poly-para-xylylene and has the following repeating structural unit shown below:

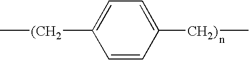

Parylene N has a high dielectric strength and provides a dielectric constant that is independent of frequency. Parylene N is adapted to be used at temperatures exceeding 220° C. Parylene C is poly-monochloro-para-xylene, and has the following repeating structural unit shown below:

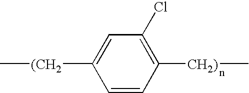

Parylene C provides a combination of physical and electrical properties including low permeability to moisture and corrosive gasses. Both Parylene N and Parylene C comply with the United States Pharmacopeia's (USP) Class IV biological testing requirements and are approved for use in medical applications by the Food and Drug Administration (FDA). A third form of parylene, Parylene D, is also available. Parylene D exhibits greater thermal stability than Parylene N or Parylene C. Parylene D has the following repeating structural unit shown below.

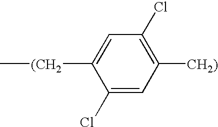

Fluorinated poly xylylene based polymers can also be used. An exemplary fluorinated poly xylylene based polymer includes Parylene HT®, also known as Parylene F. Parylene HT® is commercially available from Specialty Coating Systems located on the World Wide Web at www.scscoatings.com. Parylene HT® has the repeating structural unit shown below.

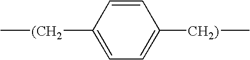

Parylene HT® has a lower dielectric constant than the other parylene variants and offers greater thermal stability. The lower dielectric constant coupled with the higher thermal stability may make Parylene HT® useful in MRI compatible applications. Additionally, Parylene HT® has a low coefficient of friction making it useful as a lubricous coating.

A table listing some of the properties of Parylene N, Parylene C, Parylene D, and Parylene HT® is provided in Table 1.

TABLE 1

| | Parylene | Parylene | | Parylene |
|---|---|---|---|---|
| | N | C | Parylene D | HT ® |
| Tensile Strength, psi | 6,500 | 10,000 | 11,000 | 7,500 |
| Dielectric Strength, | 7,000 | 6,800 | 5,500 | 5,600 |
| short time | | | | |
| (Volts/mil at 1 mil) | | | | |
| Dielectric Constant: | 2.65 | 3.15 | 2.84 | 2.2 |
| 60 Hz | | | | |
| Coefficient of | 0.25 | 0.29 | 0.33 | 0.14 |
| Friction: Static | | | | |
| Coefficient of | 0.25 | 0.29 | 0.31 | 0.13 |
| Friction: Dynamic | | | | |
| Gas Permeability*: | | | | |
| Nitrogen | 7.7 | .095 | 4.5 | — |
| Oxygen | 30 | 7.1 | 32 | 23.5 |
| Carbon Dioxide | 214 | 7.7 | 13 | — |
| Hydrogen Sulfide | 795 | 13 | 1.45 | — |
| Sulphur Dioxide | 1,890 | 11 | 4.75 | — |
| Chlorine | 74 | 0.35 | 0.55 | — |
| Moisture Vapor | 1.50 | 0.14 | 0.25 | <0.1 |
| Transmission | | | | |
| Rate** | | | | |
| Water Absorption | <0.1 | <0.1 | — | <0.1 |
| (%) | | | | |

*cm³-mil/100 in²-24 hr-atm (23° C.)
**g-mil/100 in²-24 hr, 37° C., 90% RH
1 mil = 1/1000 in. = 25.4 microns

As further described below with reference to FIGS. 2-6, in one embodiment, the parylene coating is disposed over individual conductive wires or filaments used to form the conductor 32 such that the individual wires or filaments are separately coated. According to another embodiment, the parylene coating may be disposed over the outer periphery of one or more conductors and/or conductive filaments forming the conductors. According to yet another embodiment, the parylene coating separates or is otherwise disposed between a first conductor and a second conductor.

FIG. 2A is a side schematic view of a portion of the lead body 12 including a coiled conductor 36 having two filaments 38a and 38b. FIG. 2B is an end, cross-sectional view of one filament, 38a or 38b, according to an embodiment of the present invention. As shown in FIG. 2A, the coiled conductor 36 includes a parylene coating 40 covering an outer periphery of the coiled conductor 36, according to an exemplary embodiment of the present invention. According to a further embodiment, as shown in FIG. 2B, each conductive filament 38a,b includes a conductive wire core 42 surrounded by a parylene coating 44, such that each filament 38a,38b is separately insulated from the other. Although the embodiment shown in FIGS. 2A-2B includes Parylene layers 40 and 44, each layer could be utilized independently as well.

FIG. 3 is an end cross-sectional view of the lead body 12 including a plurality of cable conductors 46, each having a plurality of conductive filaments 48. According to one embodiment, a parylene coating 50 is provided over the outer periphery of the cable conductors 46 and such that the coating 50 extends from substantially the proximal end 16 to the distal end 20 of the lead body 12. According to a further embodiment, each individual conductive filament 48 forming the cable conductor 46 includes a parylene coating such that each individual conductive filament 48 is separately insulated from the other. According to some embodiments, the coating covering the outer periphery of the cable conductors and the coating covering the individual wires or filaments may be used alone or in combination with one another.

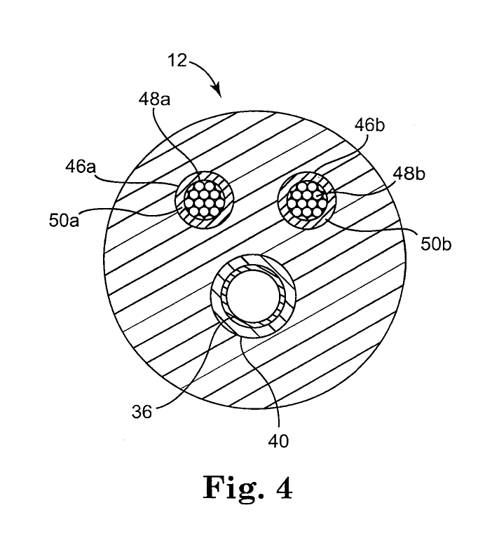

According to some embodiments of the present invention, the lead body 12 can include one or more coiled conductors 36 in combination with one or more cable conductors 46a, 46b. FIG. 4 is an end cross-sectional view of a lead body 12 including a coiled conductor 36 in combination with two cable conductors 46a and 46b, according to an exemplary embodiment of the present invention. According to one embodiment, the coiled conductor 36 includes a parylene coating 40 covering an outer periphery of the coiled conductor 36. According to a further embodiment, the filaments forming the coiled conductor 36 include a conductive wire core surrounded by a parylene coating, such that each filament is separately insulated from the other. Each of the cable conductors 46a and 46b includes a plurality of conductive filaments 48a, 48b. According to one embodiment of the present invention, the plurality of conductive filaments each include a parylene coating such that they are separately insulated from one another. According to another embodiment, a parylene coating 50a, 50b is provided over the outer periphery of the cable conductors 46a, 46b and extends from substantially the proximal end 16 to the distal end 20 of the lead body 12.

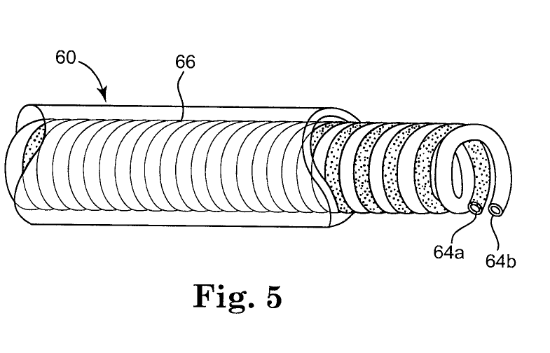

According to a further embodiment of the present invention, as shown in FIG. 5, the lead body 12 includes a coiled conductor 60 having a co-radial configuration. In this embodiment, each individual conductor wire or filament 64a, 64b is separately coated with parylene, and is then wound together in parallel to form a single coil. Additionally, the co-radial coiled conductor 60 includes at least one layer of outer insulation coating 66 covering the outer periphery of the co-radial coiled conductor from substantially the proximal end 16 to the distal end 20 of the lead body 12.

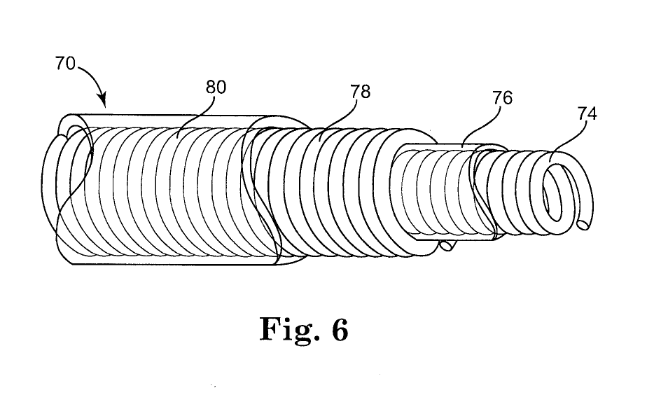

According to another further embodiment of the present invention, as shown in FIG. 6, the lead body 12 includes a coiled conductor 70 having a co-axial configuration. The co-axial coiled conductor 70 includes an inner coiled conductor 74 separated from an outer coiled conductor 78 by a parylene coating 76. Additionally, the co-axial coiled conductor also includes an outer parylene coating 80.

A non-porous, pin-hole free parylene coating may prevent metal ion migration, thus protecting the lead body 12 from degradation and/or deterioration. Degradation of the lead body 12 can occur from corrosion and/or oxidation of the conductor resulting from bodily fluid coming into contact with the conductor. Deterioration of the lead body 12 can lead result from the breakdown of the polymer used to form the lead body 12. Breakdown of the polymeric material used to form the lead body 12 can make the lead body 12 more susceptible to physical, chemical, and mechanical stresses resulting from implantation in a patient's body.

A parylene coating provided over the conductor and/or the individual conductive wire or filaments forming the conductive may improve the MRI compatibility of the lead. The parylene coating provides a low thermally conductive barrier over the conduct, and may prevent the conductor from heating or transferring heat to the surrounding tissue in response to the electromagnetic radio frequency waves generated during MRI imaging. Additionally, the parylene coating may shield the conductor from the radio frequency waves, preventing an inducting of current in the conductor.

In each of the various embodiments, as described above, other insulating polymers such as polyurethane or other similar insulating polymers and combinations thereof may be used as additional layers of insulation provided over one or more layers of the parylene coatings. In still further embodiments, one or more layers of parylene can be alternatively layered with one or more layers of another biocompatible, insulative polymer known to those of skill in the art.

According to various embodiments of the present invention, the parylene coating may be deposited onto the conductor substrate (e.g, conductor 32 or an individual wire or filament) by conventional vapor deposition polymerization techniques known to those of skill in the art. An exemplary method of depositing parylene or a parylene derivative on a substrate by vapor deposition polymerization is shown and described in U.S. Pat. No. 5,424,097, which is incorporated by reference herein. According to alternative embodiments, the parylene coating can be deposited on a substrate by plasma vapor deposition techniques known to those of skill in the art.

Vapor deposition polymerization of parylene begins with a powdered form of the dimer. Sublimated directly to a vapor and cracked to a monomeric state, the resultant parylene coating forms by spontaneous polymerization on the target substrate, such as a conductor or individual wire or filament used to form a conductor, in an evacuated, room-temperature deposition chamber. The insulation coating grows from the monomeric vapor onto the surface of the substrate one molecule at a time, facilitating the formation of a conformal, uniform layer on the substrate.

Vapor deposition polymerization facilitates the precise control of coating thickness, and facilitates the formation of a thin layer of insulation coating that is substantially non-porous and free from pinholes having a uniform thickness. Additionally, vapor deposition polymerization may facilitate the formation of thinner insulation coatings having improved physical properties while at the same time providing an effective barrier against corrosion and degradation of polymer. Additionally, the deposition of thinner layers of insulation can lead to the production of lead bodies having reduced outer diameters.

According to one embodiment, the parylene coating has a thickness ranging from about 0.1 μm to about 100 μm. According to yet another embodiment of the present invention, a parylene coating has a thickness ranging from about 0.5 μm to about 5 μm.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A medical electrical lead comprising;
an insulating outer lead body including a proximal end and a distal end;
at least one conductor extending through the outer lead body, which is configured to be connected to a pulse generator at the proximal end of the lead body and a conductor at the distal end of the lead body;
a non-porous, substantially pin-hole free, parylene coating disposed between at least a portion of the conductor and the outer lead body between the proximal and distal ends of the lead body, the parylene coating having a thickness ranging from about 0.1 μm to about 100 μm;

2. The medical electrical lead according to claim 1, wherein the Parylene coating includes Parylene N.

3. The medical electrical lead according to claim 1, wherein the Parylene coating includes Parylene C.

4. The medical electrical lead according to claim 1, wherein the Parylene coating includes Parylene D.

5. The medical electrical lead according to claim 1, wherein the Parylene coating includes Parylene HT®.

6. The medical electrical lead according to claim 1, wherein the at least one conductor includes a plurality of conductive filaments, and parylene coatings are disposed discreetly over each conductive filament.

7. The medical electrical lead according to claim 4 wherein an outer parylene coating is disposed over an outer surface of the conductor.

8. The medical electrical lead according to claim 1, wherein the at least one conductor is a coiled conductor having a co-radial configuration.

9. The medical electrical lead according to claim 1, wherein the conductor is a co-axial coiled conductor having a first coiled conductor and a second coiled conductor, wherein the parylene coating is disposed over the outer periphery of first coiled conductor.

10. The medical electrical lead according to claim 1, wherein the conductor is a cable conductor.

11. The medical electrical lead according to claim 1 wherein the Parylene coating has a dielectric constant of less than about 3.25.

12. The medical electrical lead according to claim 1 wherein the Parylene coating a moisture vapor transmission rate of equal to or less than about 1.75 g-mil/100 in².
